(19) United States
(12) Patent Application Publication
(10) Pub. No.: US 2024/0277586 A1
(43) Pub. Date: Aug. 22, 2024

(54) HEXAGONAL BORON NITRIDE POWDER AND METHOD FOR PRODUCING SAME, AND COSMETIC PREPARATION AND METHOD FOR PRODUCING SAME

(71) Applicant: Denka Company Limited, Tokyo (JP)

(72) Inventor: Ryuki MATSUI, Tokyo (JP)

(73) Assignee: Denka Company Limited, Tokyo (JP)

(21) Appl. No.: 18/567,433

(22) PCT Filed: Jun. 16, 2021
(86) PCT No.: PCT/JP2021/022906

§ 371 Date: Dec. 6, 2023

(51) Int. Cl. A61K 8/19; A61K 8/02; A61Q 19/00; C01B 21/064

(52) U.S. Cl. CPC A61K 8/19; A61K 8/022; A61Q 19/00; C01B 21/0646; C01B 21/0648; A61K 2800/10; A61K 2800/805; C01P 2002/76

(57) ABSTRACT

A hexagonal boron nitride powder, in which an attenuation rate of a positive charge is higher than an attenuation rate of a negative charge when the attenuation rates of the positive and negative charges determined through charge attenuation measurement are compared with each other. A method for producing a hexagonal boron nitride powder includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; and a firing step of heating and firing a mixed powder containing the calcined product and an aid at 1900° C. to 2100° C. for 10 to 50 hours in an atmosphere of an inert gas, ammonia gas, or a mixed gas thereof.

### TECHNICAL FIELD

The present disclosure relates to a hexagonal boron nitride powder and a method for producing the same, and a cosmetic preparation and a method for producing the same.

### BACKGROUND ART

Boron nitride has lubricity, high thermal conductivity, insulation properties, and the like, and is used in a wide range of applications, including solid lubricants, mold release agents, fillers for resins and rubber, raw materials for a cosmetic preparation (also called a cosmetic), and insulating sintered bodies with heat resistance.

Examples of functions of the hexagonal boron nitride powder mixed into the cosmetic preparation include improving slipperiness, spreadability, and concealability of the cosmetic preparation, and imparting glossiness to the cosmetic preparation. In particular, the hexagonal boron nitride powder has superior slipperiness compared to a talc powder and a mica powder, which have similar functions, and are therefore widely used in the cosmetic preparation that requires excellent slipperiness. Patent Literature 1 proposes that the ratio of shear stress to a pressing force be within a predetermined numerical range to improve slipperiness.

### CITATION LIST

### Patent Literature

[Patent Literature 1] Japanese Unexamined Patent Publication No. 2019-43792

### SUMMARY OF INVENTION

### Technical Problem

A hexagonal boron nitride powder may form agglomerated lumps due to factors such as moisture. There is a concern that agglomeration will reduce flowability and impair slipperiness and handleability. Therefore, the present disclosure provides a hexagonal boron nitride powder that can suppress agglomeration, and a method for producing the same. In addition, the present disclosure provides a cosmetic preparation which suppresses agglomeration and has excellent spreadability using the above-described hexagonal boron nitride powder, and a method for producing the same.

### Solution to Problem

According to an aspect of the present disclosure, there is provided a hexagonal boron nitride powder, in which an attenuation rate of a positive charge is higher than an attenuation rate of a negative charge when the attenuation rates of the positive and negative charges determined through charge attenuation measurement are compared with each other. The hexagonal boron nitride powder may be electrically charged due to, for example, friction between particles and friction with an inner wall of a storage container. Here, when a positive charge is generated, the polarity of oxygen atoms in water molecules present in atmospheric air is negative, and therefore a hexagonal boron nitride powder positively charged by moisture in atmospheric air agglomerates. However, in the above-described hexagonal boron nitride powder, the attenuation rate of the positive charge is higher than the attenuation rate of the negative charge, and therefore, the positive charge is quickly attenuated. Accordingly, agglomeration due to moisture in atmospheric air can be suppressed.

The ratio of the attenuation rate of the positive charge to the attenuation rate of the negative charge of the above-described hexagonal boron nitride powder may be 1.5 or less. This reduces the difference in the attenuation rate between the positive charge and the negative charge, making it possible to suppress a charge deviation. Accordingly, agglomeration caused by intervention of not only water molecules but also other molecules can be suppressed.

The above-described hexagonal boron nitride powder may be used as a raw material for a cosmetic preparation. Since agglomeration of the above-described hexagonal boron nitride powder is suppressed, the hexagonal boron nitride powder has excellent spreadability. Accordingly, it is suitable for use as a raw material for the cosmetic preparation.

A method for producing a hexagonal boron nitride powder according to an aspect of the present disclosure provides a method for producing a hexagonal boron nitride powder, the method including: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; a firing step of heating and firing a mixed powder containing the calcined product and an aid at 1900° C. to 2100° C. for 10 to 50 hours in an atmosphere of an inert gas, ammonia gas, or a mixed gas thereof; and a purification step of pulverizing, washing, and drying the fired product obtained in the firing step to obtain a hexagonal boron nitride powder, in which an attenuation rate of a positive charge is higher than an attenuation rate of a negative charge when the attenuation rates of the positive and negative charges determined through charge attenuation measurement are compared with each other.

The above-described production method includes the calcination step of performing firing at a temperature lower than that in the firing step, thereby making it possible to form hexagonal boron nitride with a small particle size and low crystallinity. In the firing step, by performing firing at 1900° C. to 2100° C. using an aid, it is possible to reduce the number of functional groups such as hydroxyl groups on the surface of the hexagonal boron nitride particles while increasing crystallinity of the hexagonal boron nitride. This makes it possible to obtain a hexagonal boron nitride powder which is less likely to be charged and of which a charge is quickly attenuated even if the powder is charged. Such a hexagonal boron nitride powder can suppress agglomeration due to static electricity. In addition, in the hexagonal boron nitride powder, an attenuation rate of a positive charge is higher than an attenuation rate of a negative charge when the attenuation rates of the positive and negative charges determined through charge attenuation measurement are compared with each other. For this reason, the positive charge is quickly attenuated. Accordingly, agglomeration due to moisture in atmospheric air can be suppressed.

A cosmetic preparation according to an aspect of the present disclosure includes the above-described hexagonal boron nitride powder. The above-described hexagonal boron nitride powder can suppress agglomeration due to factors such as moisture in atmospheric air. The cosmetic preparation containing such a hexagonal boron nitride powder has excellent spreadability.

A method for producing a cosmetic preparation according to an aspect of the present disclosure includes producing a cosmetic preparation using the hexagonal boron nitride powder obtained through any of the above-described production methods as a raw material. The hexagonal boron nitride powder obtained through the above-described production method can suppress agglomeration due to factors such as moisture in atmospheric air. For this reason, the cosmetic preparation produced using such a hexagonal boron nitride powder as a raw material has excellent spreadability.

### Advantageous Effects of Invention

According to the present disclosure, it is possible to provide a hexagonal boron nitride powder that can suppress agglomeration, and a method for producing the same. In addition, according to the present disclosure, it is possible to provide a cosmetic preparation which suppresses agglomeration and has excellent spreadability using the above-described hexagonal boron nitride powder, and a method for producing the same.

### DESCRIPTION OF EMBODIMENTS

Hereinafter, embodiments of the present disclosure will be described. However, the following embodiments are merely examples for describing the present disclosure and are not intended to limit the present disclosure to the following contents.

In a hexagonal boron nitride powder according to the present embodiment, an attenuation rate of a positive charge is higher than an attenuation rate of a negative charge when the attenuation rates of the positive and negative charges determined through charge attenuation measurement are compared with each other. In such a hexagonal boron nitride powder, the positive charge is attenuated more quickly than the negative charge. Accordingly, agglomeration caused by hydrogen bonding between oxygen atoms of water molecules and positive charges of the hexagonal boron nitride powder can be suppressed. The ratio of the attenuation rate of the positive charge to the attenuation rate of the negative charge may be greater than 1 and 1.5 or less. When this ratio approaches 1, the difference in the attenuation rate between the positive charge and the negative charge becomes small, and static electricity on the particle surface can be quickly removed.

The charge attenuation measurement in the present disclosure is performed in accordance with JIS C61340-2-1:2006 using a commercially available measurement device and is also referred to as electrostatic charge dissipativity measurement. Examples of measurement devices include NS-D100 (product name) manufactured by Nano Seeds Corporation. Using a surface potential attenuation curve obtained through this measurement, an attenuation rate (α) is calculated from the following equation.

$$V = {V}_{0} exp ( - a \sqrt{t} ) \qquad [ Equation 1 ]$$

In the equation, t represents an attenuation time, V represents a surface potential at an attenuation time t, V₀ represents an initial surface potential, and α represents an attenuation rate. The attenuation rate α can be determined by charging a hexagonal boron nitride powder in a corona discharge of a predetermined potential difference. The maximum value of the attenuation time t is set to 600 seconds, and the surface potential V up to 600 seconds is measured. α is determined by exponentially approximating the relationship between an initial surface potential V₀ value and a surface potential V value at a predetermined attenuation time t. The above-described measurement is performed by charging the hexagonal boron nitride powder with a positive charge and a negative charge. At this time, attenuation rates of the positive charge and the negative charge are determined assuming that the amount of charge is the same for both. The absolute value of the difference in the attenuation rate between the positive charge and the negative charge may be less than 0.005 or may be less than 0.003.

The hexagonal boron nitride powder according to the present embodiment is less likely to form agglomerated lumps, and therefore has excellent slipperiness and handleability. For this reason, it can be suitably used for various applications. For example, it is used in a releasing agent, an underlay powder, and the like. In addition, this hexagonal boron nitride powder has excellent spreadability when applied to a medium (such as human skin) due to suppressed agglomeration. For this reason, it is suitable for use as a raw material for a cosmetic preparation, for example. That is, the present disclosure can also provide a method of using a hexagonal boron nitride as a raw material for a cosmetic preparation.

A cosmetic preparation according to one embodiment contains the hexagonal boron nitride powder described above. Of the static electricity generated on the surface, this hexagonal boron nitride powder can reduce positive charges more quickly than negative charges. For this reason, the hexagonal boron nitride powder has suppressed agglomeration due to moisture, and therefore has excellent spreadability.

Examples of a cosmetic preparation include foundation (powder foundation, liquid foundation, and cream foundation), face powder, point makeup, eye shadow, eyeliner, nail polish, lipstick, cheek rouge, and mascara. Among these, the hexagonal boron nitride powder is particularly well suited for foundation and eye shadow. The content of the hexagonal boron nitride powder in a cosmetic preparation is, for example, 0.1 to 70 mass %. The Cosmetic preparation can be produced through well-known methods. A method for producing a cosmetic preparation includes, for example, a step of formulating and mixing a hexagonal boron nitride powder with other raw materials.

A method for producing a hexagonal boron nitride powder according to one embodiment includes: a calcination step of firing a raw material powder containing a boron-containing compound powder and a nitrogen-containing compound powder at 600° C. to 1300° C. in an inert gas atmosphere, an ammonia gas atmosphere, or an atmosphere of mixed gas thereof to obtain a calcined product containing at least one selected from the group consisting of low crystalline hexagonal boron nitride and amorphous hexagonal boron nitride; a firing step of heating a mixed powder containing the calcined product and an aid at 1900° C. to 2100° C. for 10 to 50 hours in an atmosphere of an inert gas and/or ammonia gas to obtain a fired product; and a purification step of pulverizing, washing, and drying the fired product to obtain a dry powder.

Examples of boron-containing compounds include boric acid, boron oxide, and borax. Examples of nitrogen-containing compounds include cyandiamide, melamine, and urea. The molar ratio between boron atoms and nitrogen atoms in a raw material powder containing boron-containing compound powder and a nitrogen-containing compound powder may be boron atoms:nitrogen atoms=2:8 to 8:2, or 3:7 to 7:3. The raw material powder may contain components other than the above-described compounds. The raw material powder may contain carbonates such as lithium carbonate and sodium carbonate as an aid. In addition, the raw material powder may contain a reducing substance such as carbon.

The raw material powder containing the above-described components is calcined with, for example, an electric furnace in an inert atmosphere such as nitrogen gas, helium gas, or argon gas, an ammonia atmosphere, or a mixed gas atmosphere in which these are mixed. The calcination temperature may be 600° C. to 1300° C., 800° C. to 1200° C., or 900° C. to 1100° C. The calcination time may be, for example, 0.5 to 5 hours or 1 to 4 hours.

The calcined product obtained through calcining contains at least one selected from the group consisting of low crystalline hexagonal boron nitride and amorphous hexagonal boron nitride. In the calcination step, the reaction of boron nitride is allowed to progress at a lower temperature than in the firing step to be described below. For this reason, grain growth can be suppressed, and the particle diameter of a boron nitride powder finally obtained can be reduced. In addition, the specific surface area of the hexagonal boron nitride powder can be increased.

Next, the obtained calcined product is formulated and mixed with an aid to obtain a mixed powder. Examples of aids include borates such as sodium borate and carbonates such as sodium carbonate, calcium carbonate, and lithium carbonate. The formulation amount of the aid may be 2 to 20 parts by mass or 2 to 8 parts by mass based on 100 parts by mass of the calcined product containing hexagonal boron nitride. Such a mixed powder is fired in, for example, an electric furnace in an inert atmosphere such as nitrogen gas, helium gas, or argon gas, an ammonia atmosphere, or a mixed gas atmosphere in which these are mixed.

In the firing step, production and crystallization of boron nitride progress in the presence of the aid. As a result, the crystallinity of the boron nitride contained in the calcined product can be enhanced. The firing temperature is 1900° C. to 2100° C., or may be 1950° C. to 2050° C. The firing time may be, for example, 10 to 50 hours or 20 to 40 hours. By performing firing under such conditions, functional groups such as hydroxyl groups present on the particle surface can be scattered. This makes it possible to obtain a hexagonal boron nitride powder which is less likely to be charged with static electricity and in which the attenuation rate of the positive charge is higher than the attenuation rate of the negative charge.

If the firing temperature becomes too low, the amount of functional groups such as hydroxyl groups on the surface of the hexagonal boron nitride tends to increase. As the amount of the above-described functional groups on the surface of the hexagonal boron nitride increases, the hexagonal boron nitride is likely to be charged with static electricity and the attenuation rate of the negative charge becomes higher than the attenuation rate of the positive charge. In addition, the ratio of the attenuation rate of the positive charge to the attenuation rate of the negative charge becomes too small. This tends to easily cause agglomeration. The same trend is observed even when the firing time is too short. On the other hand, if the firing temperature is too high, the crystal growth of hexagonal boron nitride proceeds too much, whereby primary particles tend to agglomerate. The same trend is observed even when the firing time is too long.

The fired product obtained in the firing step may be pulverized with a usual pulverization device. The pulverized powder may contain impurities in addition to hexagonal boron nitride. Examples of impurities include a residual aid and a water-soluble boron compound. In the purification step, such impurities are reduced through washing. After washing, solid-liquid separation and drying are performed to obtain a dry powder. Examples of washing liquids used for washing include water, an aqueous solution containing an acidic substance, an organic solvent, and a mixed liquid of an organic solvent and water. From the viewpoint of avoiding secondary contamination of impurities, water having an electric conductivity of 1 mS/m or less may be used. Examples of acidic substances include inorganic acids such as hydrochloric acid and nitric acid. Examples of organic solvents include water-soluble organic solvents such as methanol, ethanol, propanol, isopropyl alcohol, and acetone. The washing method is not particularly limited. For example, the pulverized powder may be immersed in a washing liquid and stirred and washed, or the pulverized powder may be washed by spraying a washing liquid.

After the completion of washing, the washing liquid may be subjected to solid-liquid separation through decantation or using a suction filter, a pressure filter, a rotary filter, a sedimentation separator, or a combination thereof. A dry powder may be obtained by drying the separated solid content in a usual dryer. Examples of dryers include a shelf-type dryer, a fluidized bed dryer, a spray dryer, a rotary-type dryer, a belt-type dryer, and a combination thereof. After drying, for example, classification with a sieve may be performed to remove coarse particles.

The above-described hexagonal boron nitride powder can be obtained in this manner. In the hexagonal boron nitride powder obtained through the above-described production method, the attenuation rate of the positive charge may be higher than the attenuation rate of the negative charge when the attenuation rates of the positive and negative charges determined through charge attenuation measurement are compared with each other. In addition, the ratio of the attenuation rate of the positive charge to the attenuation rate of the negative charge may be greater than 1 and 1.5 or less.

The description regarding the above-described embodiment of the hexagonal boron nitride powder can also be applied to the method for producing a hexagonal boron nitride powder. The method for producing a hexagonal boron nitride powder is not limited to the above-described embodiment. For example, after the firing step, a crushing step of crushing a hexagonal boron nitride powder using a homogenizer applying ultrasound vibration may be performed.

Some embodiments of the present disclosure have been described above, but the present disclosure is not limited to any of the above-described embodiments.

### EXAMPLES

The contents of the present disclosure will be described in more detail with reference to examples and a comparative example, but the present disclosure is not limited to the following examples.

### Example 1

### [Preparation of Hexagonal Boron Nitride Powder]

### <Calcination Step>

100.0 g of a boric acid powder (purity of 99.8 mass % or more, manufactured by Kanto Chemical Co., Inc.) and 90.0 g of a melamine powder (purity of 99.0 mass % or more, manufactured by Wako Pure Chemical Industries, Ltd.) were mixed with each other with an alumina mortar for 10 minutes to obtain a mixed raw material. The mixed raw material after drying was placed in a container made of hexagonal boron nitride and placed in an electric furnace. The temperature was raised from room temperature to 1000° C. at a rate of 10° C./min while circulating nitrogen gas in the electric furnace. After holding the temperature at 1000° C. for 2 hours, the heating was stopped and the mixture was allowed to cool naturally. The electric furnace was open at a point in time when the temperature became 100° C. or lower. In this manner, a calcined product containing low crystalline hexagonal boron nitride was obtained.

### <Firing Step>

3.0 g of sodium carbonate (purity of 99.5 mass % or more) was added to 100.0 g of the calcined product and mixed with each other with an alumina mortar for 10 minutes. The mixture was placed in the above-described electric furnace. The temperature was raised from room temperature to 2000° C. at a rate of 10° C./min while circulating nitrogen gas in the electric furnace. After holding the temperature at 2000° C. for 30 hours, the heating was stopped and the mixture was allowed to cool naturally. The electric furnace was open at a point in time when the temperature became 100° C. or lower. The obtained fired product was collected and pulverized with alumina mortar for 3 minutes to obtain a coarse powder of hexagonal boron nitride.

### <Purification Step>

In order to remove impurities contained in a coarse powder of hexagonal boron nitride, 30 g of the coarse powder was put into 500 g (concentration of nitric acid: 5 mass %) of dilute nitric acid and stirred at room temperature for 60 minutes. After stirring, solid-liquid separation was performed through suction filtration, and washing was performed by replacing the liquid with water (electric conductivity of 1 mS/m) until the filtrate became neutral. After washing, the solid was dried with a dryer at 120° C. for 3 hours to obtain a dry powder. The obtained dry powder was used as a hexagonal boron nitride powder of Example 1.

### [Evaluation of Hexagonal Boron Nitride Powder]

### <Evaluation of Appearance>

The appearance of the obtained hexagonal boron nitride powder was observed. As a result, it was confirmed that the hexagonal boron nitride powder did not agglomerate and had excellent flowability.

### <Evaluation of Charge Attenuation>

The charge attenuation of the hexagonal boron nitride powder produced in Example 1 was measured with an electrostatic dissipativity measurement device (manufactured by Nano Seeds Corporation, product name: NS-D100) in accordance with JIS C61340-2-1:2006. The measurement was carried out in a constant-humidity and constant-temperature chamber adjusted to a temperature of 23° C. and a relative humidity of 50%. The charging time for positive and negative charges was 1 second, the sampling frequency was 1 Hz, and the measurement time was 600 seconds. The distance from a sensor to the powder surface was set to about 1 mm. The measurement sample was placed in a 5 cm×5 cm×0.4 cm (10 cm³) cell on a sample plate and charged by a corona discharge. Charging was performed with a positive charge and a negative charge. After charging, the measurement sensor was driven, and the attenuation of surface potential was measured. An initial surface potential V₀ and a surface potential (final surface potential V₁) until a measurement time of 600 seconds was elapsed were determined for each of the positive charge and the negative charge from the determined surface potential attenuation curve. The measurement interval was 1 second. An attenuation rate α was determined by exponentially approximating the relationship between an initial surface potential V₀ value and a surface potential V value at a predetermined attenuation time t using the following equation.

$$V = {V}_{0} exp ( - a \sqrt{t} ) \qquad [ Equation 2 ]$$

In the above equation, t represents an attenuation time, V represents a surface potential at an attenuation time t, V₀ represents an initial surface potential, and a represents an attenuation rate. The results are shown in Table 2.

### <Evaluation for Spreadability>

0.2 g of the hexagonal boron nitride powder was placed on one end of an artificial skin (length×width=10 mm×50 mm). The hexagonal boron nitride powder was spread along the surface of the artificial skin in the vertical direction using a spatula so that the hexagonal boron nitride powder was applied to the surface. Image analysis was performed using commercially available image analysis software (WinROOF) to determine the ratio of the coating area of the hexagonal boron nitride powder to the total area of the artificial skin. The larger this area proportion, the better the spreadability. The evaluation criteria for spreadability were as shown in Table 1 depending on the area proportion. The evaluation results for spreadability are as shown in Table 2.

### Example 2

A hexagonal boron nitride powder was prepared in the same manner as in Example 1 except that the holding time in the firing step was set to 15 hours. Then, the hexagonal boron nitride powder was evaluated in the same manner as in Example 1. The evaluation results are as shown in Table 2. The appearance of the obtained hexagonal boron nitride powder was observed. As a result, it was confirmed that the hexagonal boron nitride powder hardly agglomerated and had excellent flowability.

### Example 3

A hexagonal boron nitride powder was prepared in the same manner as in Example 1 except that the holding temperature in the firing step was set to 1900° C. Then, the hexagonal boron nitride powder was evaluated in the same manner as in Example 1. The evaluation results are as shown in Table 2. The appearance of the obtained hexagonal boron nitride powder was observed. As a result, it was confirmed that the hexagonal boron nitride powder hardly agglomerated and had excellent flowability.

### Comparative Example 1

A hexagonal boron nitride powder was prepared in the same manner as in Example 1 except that the firing temperature in the firing step was set to 1700° C. The powder was evaluated in the same manner as in Example 1. The results are as shown in Table 2. The appearance of the obtained hexagonal boron nitride powder was observed. As a result, the hexagonal boron nitride powder agglomerated.

TABLE 1

| Area proportion | Determination |
|---|---|
| 95% or more | Very good |
| 80% or more and less than 95% | Good |
| 60% or more and less than 80% | Fair |
| 40% or more and less than 60% | Poor |
| Less than 40% | Very poor |

TABLE 2

| | | | | Attenu- | Proportion | |
|---|---|---|---|---|---|---|
| | | V₀ | V₁ | ation rate | of attenu- | Spread- |
| | | [kV] | [kV] | (α) | ation rate | ability |
| Example 1 | Positive | 1.11 | 1.06 | 0.0017 | 1.11 | Very |
| | charge | | | | | good |
| | Negative | −1.18 | −0.13 | 0.0016 | | |
| | charge | | | | | |
| Example 2 | Positive | 1.12 | 0.95 | 0.0069 | 1.37 | Good |
| | charge | | | | | |
| | Negative | −1.21 | −1.07 | 0.0050 | | |
| | charge | | | | | |
| Example 3 | Positive | 1.07 | 0.90 | 0.0074 | 1.48 | Good |
| | charge | | | | | |
| | Negative | −1.24 | −1.08 | 0.0050 | | |
| | charge | | | | | |
| Comparative | Positive | 1.00 | 0.69 | 0.0110 | 0.64 | Very |
| Example 1 | charge | | | | | poor |
| | Negative | −1.12 | −0.57 | 0.0170 | | |
| | charge | | | | | |

The column “Proportion of attenuation rate” in Table 2 shows a ratio of an attenuation rate of a positive charge to an attenuation rate of a negative charge. In the hexagonal boron nitride powders of Examples 1 to 3, the attenuation rate of a positive charge was higher than the attenuation rate of a negative charge. When observing the appearance, Comparative Example 1 formed agglomerated lumps, whereas Examples 1 to 3 clearly had fewer agglomerated lumps than Comparative Example 1. In addition, Examples 1 to 3 had better spreadability than Comparative Example 1.

### INDUSTRIAL APPLICABILITY

According to the present disclosure, it is possible to provide a hexagonal boron nitride powder in which agglomeration is suppressed, and a method for producing the same. In addition, a cosmetic preparation which has excellent spreadability and in which agglomeration is suppressed using the above-described hexagonal boron nitride powder is provided.

1. A hexagonal boron nitride powder,
wherein an attenuation rate of a positive charge is higher than an attenuation rate of a negative charge when the attenuation rates of the positive and negative charges determined through charge attenuation measurement are compared with each other.

2. The hexagonal boron nitride powder according to claim 1,
wherein a ratio of the attenuation rate of the positive charge to the attenuation rate of the negative charge is 1.5 or less.

3. The hexagonal boron nitride powder according to claim 1, which is for a raw material for a cosmetic preparation.

4. A method for producing a hexagonal boron nitride powder, the method comprising:
a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride;
a firing step of heating and firing a mixed powder containing the calcined product and an aid at 1900° C. to 2100° C. for 10 to 50 hours in an atmosphere of an inert gas, ammonia gas, or a mixed gas thereof; and
a purification step of pulverizing, washing, and drying a fired product obtained in the firing step to obtain a hexagonal boron nitride powder,
wherein an attenuation rate of a positive charge is higher than an attenuation rate of a negative charge when the attenuation rates of the positive and negative charges determined through charge attenuation measurement are compared with each other.

5. A cosmetic preparation comprising:
the hexagonal boron nitride powder according to claim 1.

6. A method for producing a cosmetic preparation, the method comprising:
producing the cosmetic preparation using the hexagonal boron nitride powder obtained through the method for producing according to claim 4 as a raw material.
